(19) United States
(12) Patent Application Publication
(10) Pub. No.: US 2011/0159061 A1
(43) Pub. Date: Jun. 30, 2011

(54) WIPE ARTICLE COMPRISING LOTION COMPOSITION COMPRISING OMEGA-6 FATTY ACID AND METHOD OF IMPROVING SKIN BARRIER FUNCTION

(76) Inventor: Raphael WARREN, Amberley Village, OH (US)

(21) Appl. No.: 12/974,731

(22) Filed: Dec. 21, 2010

Related U.S. Application Data
(60) Provisional application No. 61/291,069, filed on Dec. 30, 2009.

(51) Int. Cl. A61K 8/92; A61K 8/02; A61Q 19/00

(52) U.S. Cl. 424/401; 514/560; 514/458; 514/464

(57) ABSTRACT

A wipe article comprising a lotion composition comprising omega-6 fatty acid. A method of improving skin barrier function of vulvar skin comprising the step of contacting the vulvar skin with a wipe article, wherein omega-6 fatty acid is disposed on the surface of the wipe article.

### CROSS REFERENCE TO RELATED APPLICATION

This application claims the benefit of U.S. Provisional Application No. 61/291,069, filed Dec. 30, 2009.

### FIELD OF THE INVENTION

The present invention relates to a wipe article comprising a lotion composition comprising omega-6 fatty acid and a method of improving skin barrier function of semi-occluded skin by contacting the semi-occluded skin with the wipe article.

### BACKGROUND OF THE INVENTION

Disposable absorbent articles, such as diapers, training pants, and catamenial devices having lotioned topsheets are known. Lotions of various types are known to provide various skin benefits, such as prevention or treatment of diaper rash. These lotions can be applied to the topsheet of absorbent articles, for example, and can be transferred to the skin of the wearer during use. Similarly, wipe articles containing lotions are known.

The application of lotion compositions to topsheets of absorbent articles have been primarily directed to baby diapers, with the benefit provided being better skin health for the bottom of the baby. Little attention has been directed to the unique problems associated with the skin of an adult woman when wearing a feminine hygiene product. The skin of the vulvar area of an adult woman is very different than that of a baby's bottom (or buttock skin in general). For example, the vulvar area will generally be populated with hair. It is known that adult onset hormones (i.e., estrogens, progestins, corticosteroids) influence the disposition of the epidermis and dermis, the production of lubricating skin lipids, or the skin barrier function. The vulvar skin is considerably thicker than other types of skin, with considerably more skin folds. Furthermore, hormonal changes associated with the onset of a woman's period can affect her skin sensitivity.

Independent of the menstrual cycle, vulvar skin also has an inferior skin barrier function and a high skin turnover rate comparable to those experiencing moderate skin lesions of psoriasis or those with atopic dermatitis, as compared to the reference standard, the volar forearm. Despite being a naturally humid environment (by virtue of wearing garments), a byproduct of wearing feminine hygiene products is the feeling of discomfort, skin chafing, and increased sensitivity that leads women to adapt habits such as frequent showering, frequent and costly catamenial pad changes, application of moisturizers and similar medicants. It would be desirable if a wipe article placed in contact with the vulvar area, could attenuate this discomfort. It would be even more desirable if a wipe article in contact with the vulvar area could improve vulvar skin by enabling greater resistance to environmental insults.

Accordingly there is a continuing desire for a wipe article that can improve the skin barrier function of vulvar skin.

The skin of mammals, such as humans, can be distinguished by whether it is generally exposed to the air, that is being able to transpire, or non-occluded. Alternatively the skin may be covered or occluded. Occlusion is generally understood to represent being covered by a material that does not allow the skin to transpire readily, i.e., using a plastic film. However, in many cases the skin is actually clothed using materials that allow some or limited transpiration. This type of coverage is generally termed semi-occluded. Regions of the body that are commonly described as semi-occluded include the genital area, buttocks, and underarm of the person. Physiological properties of these skin regions include hyperhydration, bacterial abundance, immune cell activation, and an inferior skin barrier. Other consequences of these physiological properties include the feeling of discomfort, skin chafing, increased sensitivity, and irritation and odor. Common adaptations for these undesirable feelings include frequent showering, use of costly powders and lotions and medicants. It would be desirable if an article in contact with the semi-occluded area could attenuate this discomfort. It would be even more desirable if an article in contact with the semi-occluded area could improve semi-occluded skin by enabling greater resistance to environmental insults.

Accordingly there is a continuing desire for a wipe article that reduces the discomfort associated with wearing absorbent articles and can improve the skin barrier function of vulvar skin.

### SUMMARY OF THE INVENTION

The present invention relates to a wipe article comprising a lotion composition comprising omega-6 fatty acid.

In one embodiment, the lotion composition comprises (a) an oil material comprising at least about 3%, by weight of the oil material, of omega-6 fatty acid, wherein the oil material has an oil stability index of at least about 10 hours, and (b) a carrier.

In another embodiment, the lotion composition comprises (a) at least about 0.003%, by weight of the lotion composition, of omega-6 fatty acid, (b) at least about 0.01%, by weight of the lotion composition, of oleic acid, and (c) a carrier.

The present invention further relates to a method of improving skin barrier function of vulvar skin, the method comprising contacting vulvar skin with a wipe article comprising omega-6 fatty acid disposed on the surface of the wipe article.

### DETAILED DESCRIPTION OF THE INVENTION

As used herein, the term “wipe article” refers to a piece of material, generally non-woven material, used to cleanse body parts. In particular, most currently available wipe articles are intended for the cleaning of the peri-anal area after defecation. Other wipe articles are available for the cleansing of the face or other body parts. The present invention focuses on wipe articles for the vulvar region. Wet-wipe articles are generally of sufficient dimension to allow for convenient handling while being small enough to be easily disposed of by the sewage system or discretely disposed of in garbage bins. The material of the wipe articles is generally soft and flexible, potentially having a structured surface enhancing its cleaning performance. The material is preferably a non-woven material, generally made of synthetic compounds. However, woven materials as well as the use of natural compounds in either woven or nonwoven materials are within the scope of the present invention. The texture and material of the wipe article are of high relevance to the performance of the wipe article. In one embodiment of the present invention the non-woven material comprises fibers selected from the group consisting of polyolefin, polyester, cellulose, rayon, polyamides, polyesteramide, polyvinyl alcohols, and combinations thereof. The substrate usable for this invention can be manufactured via any suitable process, such as but not limited to, spunlace process and preferably has a dry basis weight of between about 45 grams per square meter (gsm) and 75 gsm, more preferably between 45 gsm and 65 gsm.

The size of the wipe article can vary. The wipe article can be greater than or equal to about 4 square inches (about 25 square centimeters) in size, greater than or equal to about 9 square inches (about 50 square centimeters) in size, less than or equal to about 225 square inches (about 1,450 square centimeters) in size, between about 16 square inches (about 100 square centimeters) and about 50 square inches (about 320 square centimeters), or about 35 square inches (about 225 square centimeters) in size. Typically, each individual wipe is arranged in a folded configuration and stacked one on top of the other to provide a stack of wipes. Such folded configurations are well known to those skilled in the art and include c-folded, z-folded, quarter-folded configurations and the like. The stack of folded wipes may be placed in the interior of a container, such as a plastic tub or flexible refill bag, to provide a package of wipes for eventual sale to the consumer. Alternatively, the wipes may include a continuous strip of material which has perforations between each wipe and which may be arranged in a stack or wound into a roll for dispensing.

The wipe article can be a cleansing wipe. The wipe article can also be a hygienic cleansing wipe that may be used by the wearer to clean menses and/or other body exudates from her body. The cleaning of menses can be particularly important because when menses leaves the wearer's body, it may tend to smear over the pudendal region of the wearer's body and be retained on the wearer's skin and pubic hair. Furthermore, the menses may then dry on the skin and in the pubic hair, and make later cleansing difficult.

Without being bound by theory, it is believed that a textured wipe may further enable the ease of removal of the bodily exudates by improving the ability to grip or otherwise lift the exudates from the skin during cleansing. Any one of a number of texture elements may be useful in improving the ability to grip or otherwise lift the exudates from the skin during cleansing such as, but not limited to continuous hydro-molded elements, hollow molded element, solid molded elements, circles, squares, rectangles, ovals, ellipses, irregular circles, swirls, curly cues, cross hatches, pebbles, lined circles, linked irregular circles, half circles, wavy lines, bubble lines, puzzles, leaves, outlined leaves, plates, connected circles, changing curves, dots, honeycombs, etc. and combinations thereof. The texture elements may be hollow elements. The texture elements may be connected to each other. The texture elements may overlap each other.

Wipe articles are generally impregnated with a liquid or semi liquid composition, intended to both enhance the cleaning and to provide a smooth feeling. Generally the composition is of sufficiently low viscosity to impregnate the entire structure of the wipe article. In some other instances, the composition can be primarily present at the wipe article surface and to a lesser extent in the inner structure of the wipe article. In one optional embodiment the composition is releasably carried by the material, that is, the composition is contained either in or on a substrate and is readily releasable from the substrate by applying some force to the substrate, for example, wringing the substrate, or wiping a surface, such as a child's bottom, with the wet-wipe article. Alternatively, the wipe may also be dried after it is impregnated with the lotion creating a dry wipe. Prior to use, water is added to a dry wipe to make the lotion more readily released.

It is generally believed that the integrity of the skin barrier is related to the lipid composition of the stratum corneum, e.g., ceramides, cholesterol, triglycerides, cholesterol esters, etc. As used herein, the term “lipid” includes, but is not limited to, fats and oils and their acid analogues (i.e., fatty acids). Compositional changes, in particular to the ceramide class of lipids, feature prominently as a consequence of genetic predisposition (i.e., atopy, X-linked ichthyosis), environmental insults (i.e., irritants, winter cold/dry skin or xerosis, UV-light), or disease (i.e., psoriasis). Ceramide EOS (formerly identified as ceramide 1) has been linked with skin barrier functionality. For example, it is cross-linked to the protein envelope of the corneocyte and is believed to be the molecular ‘rivet’ that organizes the lipid lamellae. This ceramide link has led to development of topical remedies that contain lipid supplements (i.e., ceramides or ceramide derivatives) thought to be corrective for interior skin barrier activity. Despite a link between lipid composition and skin barrier, little is known of the skin surface lipid composition of semi-occluded skin, i.e., the vulvar. Despite considerable differences in skin barrier behavior, recent reports show there may be little to no meaningful difference between the superficial lipid composition of the semi-occluded vulvar skin and the non-occluded forearm skin. Indeed, data suggest that a more important factor contributing to skin barrier properties is the relative abundance of the unsaturated fatty acid that is esterified to the sphingosine base of ceramide EOS. For example, the greater the amount of linoleate the more intact is the skin barrier. Data suggest that despite no difference in the relative abundance of ceramide EOS between the vulva and the forearm there is a 75% reduction in the abundance of ω-6 fatty acid ester linoleate in the vulvar area relative to the forearm.

An interesting remedy to the inferior semi-occluded skin barrier, and thus improve skin comfort, would be to supplement this particular skin site with compositions rich in essential fatty acids or lipids, in particular, ω-6 fatty acids or fatty acid esters. As used herein, the term “essential fatty acids” means fatty acids which cannot be synthesized by the human body and must be obtained from a dietary source. Because humans lack the required enzyme to introduce carbon-carbon double bonds at carbon atoms beyond the ninth carbon atom in unsaturated fatty acids (the ninth carbon atom from the omega end of the chain), linoleic acid (an ω-6 fatty acid) and alpha linolenic acid (an ω-3 fatty acid) are essential fatty acids that must be obtained by humans from a dietary source to ensure good wellness. Many humans have been found to be deficient in essential fatty acids which can lead to numerous heath ailments and problems. It is well known that common oils such as those derived from the sunflower, low erucic rapeseed (or canola), flax (or linseed), soybean, etc. contain an abundance of unsaturated essential fatty acids, including the desirable ω-6 fatty acids. Dietary supplementation with materials rich in essential fatty acids (flax seed oil, fish oil, borage oil, evening primrose oil are believed to ameliorate skin ailments. However, dietary supplementation requiring large doses (gram per day) can be wasteful, and not necessarily reach the semi-occluded skin target. It is also well known that unsaturated fatty acids are not stable and easily oxidize. Oxidation can be promoted by multiple sources that include temperature, light, air, oxygen, moisture, and metals (in particular copper) Common sources of product making instability can include the lotion making and application process. For example, melting and mixing the lotion ingredients can require high temperatures (above the melting point of the ingredients i.e., greater than 70° C.). The lotion can stay in the tank for a considerable time (i.e., >24 hr). Another source of instability is the shelf storage of the finished product. It is not unusual for product to remain on the shelf (in the store or at home) for at least a year and, depending on geographical location, temperatures can exceed 40° C. Another source of instability can be formulations that are water or glycol based. Collectively, these factors can lead to oxidation and creation of reactive oxygen free radicals or active oxygen. This can lead to product deterioration such as discoloration (i.e., yellowing) and rancid odor. It is also known that when in contact with the skin, active oxygen can damage the skin barrier.

Accordingly there is a continuing desire for a wipe article that reduces skin discomfort associated semi-occluded skin, can improve the skin barrier function of semi-occluded skin, and can contain a lotion that contains a stable form of omega fatty acids.

The use of absorbent articles, especially for feminine hygiene purposes, in the vulvar skin area can lead to various skin problems including irritation, chafing, and the like. It has been found that vulvar skin tends to exhibit deficiencies in omega-6 fatty acid content, especially in comparison to skin in other areas of the body, such as forearm skin. This deficiency in omega-6 fatty acid can result in inferior skin barrier. Therefore, increasing the omega-6 fatty acid content of vulvar skin can help to improve skin barrier function of vulvar skin and reduce the potential for skin problems normally associated with the use of absorbent articles for feminine hygiene purposes.

To address this concern, a wipe article of the present invention comprises a lotion composition comprising omega-6 fatty acid. The lotion composition will typically comprise at least about 0.003%, from about 0.003% to about 35%, from about 0.015% to about 25%, or from about 0.06% to about 20%, by weight of the lotion composition, of omega-6 fatty acid.

The omega-6 fatty acid will typically be contained in an oil material. Therefore, in one embodiment, the lotion composition comprises an oil material comprising omega-6 fatty acid. The lotion composition will typically comprise from about 0.1% to about 70%, from about 0.5% to about 50%, or from about 2% to about 40%, by weight of the lotion composition, of the oil material. The oil material will typically comprise at least 3%, from about 3% to about 50%, or from about 5% to about 40%, by weight of the oil material, of omega-6 fatty acid.

Unsaturated fatty acids, such as omega fatty acids, tend to be instable and tend to easily oxidize. Oxidation can be promoted by multiple sources that include temperature, light, air, oxygen, moisture, and metals. See, e.g., Belitz H-D, Grosch W, and Schieberle P, Lipids In Food Chemistry 3rd ed. Springer-Verlag, Heidelberg, 2004, p. 157-242. Indeed, common sources of product making can promote instability. For example, melting and mixing the lotion composition ingredients can require high temperatures (to a temperature above the melting point of the lotion composition ingredients, e.g., greater than 70° C.). In order to melt and preserve the uniformity of a semi-solid lotion composition, it is common to heat the lotion composition application tank to high temperatures (e.g., greater than 60° C., preferably above 70° C.) with mixing. Furthermore, the lotion composition can remain in the tank for a considerable amount of time (e.g., greater than 24 hours). Another source of instability can be the shelf storage of the finished product. It is not unusual for product to remain on the shelf (in the store or at home) for at least a year and, depending on geographical location, storage temperatures can exceed 40° C. Another source of instability can result from lotion compositions that are water- or glycol-based. Collectively, these factors can lead to oxidation and creation of reactive oxygen-free radicals or active oxygen. This can lead to product deterioration such as discoloration (i.e., yellowing) and/or rancid odor. When in contact with the skin, active oxygen can damage skin barrier function.

A common measure for monitoring oxidative stability is the development of hydroperoxides (peroxide value or PV) over time. Oxidative stability can also be expressed in terms of the time required to obtain secondary oxidation products when aerating a sample at elevated temperature. A suitable measure of oxidative stability is called the Oil Stability Index (referred to herein as “OSI”). The OSI of an oil material can be measured according to the American Oil Chemical Society Oil Stability Index Method (AOCS Official Method Cd 12b-92).

In one embodiment, the oil material of the present invention is selected to have an oil stability index (“OSI”) of at least about 10 hours, at least about 14 hours, or at least about 18 hours.

It is believed that oil materials comprising relatively high levels of oleic acid tend to be more stable in the context of the present invention. In one embodiment, the oil material of the present invention comprises at least about 10%, from about 10% to about 80%, or from about 15% to about 70%, by weight of the oil material, of oleic acid. In one embodiment, the lotion composition comprises from about 0.01% to about 56%, from about 0.05% to about 40%, or from about 0.2% to about 32%, by weight of the lotion composition, of oleic acid.

It is believed that oil materials comprising relatively low levels of linolenic acid (omega-3 fatty acid) tend to be more stable in the context of the present invention. In one embodiment, the oil material of the present invention comprises less than about 10%, from about 10% to about 5%, or from about 5% to about 0%, by weight of the oil material, of linolenic acid. In one embodiment, the lotion composition comprises from about 7% to about 0%, from about 5% to about 0%, or from about 4% to about 0%, by weight of the lotion composition, of linolenic acid.

Non-limiting examples of suitable oil materials exhibiting the desired properties described herein include oleic canola Oil (Brassica campestris, B. napus, B. rapa; characterized by having an oleic content greater than 70%, e.g., hi oleic canola oil, very high oleic canola oil, or partially hydrogenated canola oil), marula kernel oil (Sclerocarya birrea), palm oil (Elaeis Guineensis Oil), palm olein, palm stearin, palm superolein, pecan oil, pumpkin seed oil, oleic safflower oil (Carthamus Tinctorius; characterized by having an oleic content of greater than about 30% and omega-6 fatty acid content of less than about 50%, e.g., hi oleic safflower oil), sesame oil (Sesamum indicum, S. oreintale), soybean oil (Glycine max, e.g., hi oleic soybean, low linolenic soybean oil, partially hydrogenated), oleic sunflower oil (Helianthus annus; characterized by having an oleic content of greater than about 40%, e.g., mid oleic sunflower or high oleic sunflower oil), and mixtures thereof. Oleic canola oil, palm oil, sesame oil, hi oleic safflower oil, hi oleic soybean oil, mid oleic sunflower oil, and high oleic sunflower oil are common plant-bred derived oils and may be also be derived from non-genetically modified organisms (non-GMO).

Non-limiting examples of oil materials are commercially-available from a number of vendors, including Cargill for partially hydrogenated soybean oil (i.e., Preference® 110W Soybean Oil or Preference® 300 Hi Stability Soybean Oil), mid oleic sunflower oil (i.e., NuSun® Mid-Oleic Sunflower Oil), high oleic sunflower oil (i.e., Clear Valley® High Oleic Sunflower Oil), high oleic canola oil, very high oleic canola, and partially hydrogenated low erucic rapeseed oil (i.e., Clear Valley® 65 High Oleic Canola Oil and Clear Valley® 75 High Oleic Canola Oil); Lambert Technology for high oleic canola oil (i.e., Oleocal C104); Arch Personal Care for marula kernel oil; Pioneer for high oleic soybean oil (i.e., Plenish®); Asoyia for low linolenic soybean oil (i.e., Ultra Low Linolenic Soybean Oil®); and Dipasa, Inc. for refined sesame oil.

It should be noted that the grade of oil material can be important as well in achieving the desired properties of the oil material as described herein. For example, the source of the oil material can be important, as the same oil (e.g. sesame oil) can exhibit a wide range of OSI values depending upon the source of the oil material.

The oil material can further comprise a blend of oils, including those described supra, as well as additional oil materials. Suitable additional oil materials can include acai berry oil, almond oil, avocado oil, beech oil, brazil nut oil, camelina sativa oil (family Brassicaceae, e.g. Camelina Sativa, Gold of Pleasure, False Flax, etc.), camellia seed oil, canola oil, carrot seed oil, cashew nut oil, caster oil, cherry kernel oil, chia oil, corn oil, cottonseed oil, hydrogenated cottonseed oil, evening primrose oil, filbert (hazelnut) oil, grapeseed oil, hemp oil, hickory nut oil, jojoba oil, kukui oil, lanolin, olive oil (Olea europaea), macadamia oil, maring a oil, meadowfoam oil, neem oil, palm kernel oil, olive oil, passionflower oil (family Passiflora, Passiflora Incarnata), peanut oil, peach kernel oil, pistachio nut oil, rapeseed oil, rice bran oil, rose hip oil, safflower oil, sorghum oil, soybean oil, sunflower seed oil, tall oil, vegetable oil, vegetable squalene, walnut oil, wheat germ oil, and mixtures thereof. The oil material of the present invention can be selected from the group consisting of camelina sativa seed oil, oleic canola oil, evening primrose oil, manila kernel oil, palm oil, palm olein, palm stearin, palm superolein, passiflora incarnata seed oil, pecan oil, pumpkin seed oil, oleic safflower oil, sesame oil, soybean oil, oleic sunflower oil, vegetable oil and mixtures thereof.

Preferred oil materials of the present invention include a mixture of vegetable oil and camelina sativa seed oil (commercially-available as Lipex° Omega 3/6 from Aarhus Karlshamn Sweden AB), a mixture of vegetable oil and passiflora incarnata seed oil (commercially-available as Lipex® Omega Passiflora from Aarhus Karlshamn Sweden AB), a mixture of vegetable oil and evening primrose oil (commercially-available as Lipex Omega EPO from Aarhus Karlshamn Sweden AB), high oleic canola oil (commercially-available as Clear Valley® 75 High Oleic Canola Oil from Cargill), or mixtures thereof.

To further enhance the stability of the oil material, certain antioxidants can be added to certain oil materials or to the lotion composition. In one embodiment, the oil material comprises from about 0.005% to about 1%, from about 0.01% to about 0.5%, or from about 0.02% to about 0.2%, by weight of the oil material, of an antioxidant. In one embodiment, the lotion composition comprises from about 0.01% to about 1%, from about 0.05% to about 0.75%, or from about 0.2% to about 0.6%, by weight of the lotion composition, of an antioxidant.

Attempts have been made to stabilize oxidatively unstable oils with antioxidants with unpredictable outcomes. See, e.g., Merrill L I, Pike O A, Ogden L V, Oxidative stability of conventional and high-oleic vegetable oils with added anti-oxidants, J Am Oil Chem Soc 85:771-776, 2008; Chu Y-H and Hsu H-F, Effect of antioxidants on peanut oil stability, Food Chemistry 66:29-34, 1999; and Isbell T A, Abbott T P, and Carlson K D, Oxidative stability index of vegetable oils in bianary mixture with meadowfoam oil, Ind Crops Products 9:115-123, 1998. Other antioxidants, such as the phenolic tert-butylhydroquinone (TBHQ), butylated hydroxytoluene (BHT), or butylated hydroxyanisole (BHA) have been reported to stabilize oils although these are known skin sensitizers and would have limited value in an absorbent product having direct contact with the skin. Furthermore, blending unstable and stable oils does not necessarily lead to acceptable oil stability profiles, however, and an undesirable consequence can be the dilution of the desirable omega-6 fatty acid below a level that is desirable.

Non-limiting examples of suitable antioxidants include α-tocopherol, β-tocopherol, γ-tocopherol, δ-tocopherol, tocotrienol, rosemary, sesamol, sesamolin, sesamin, catechin, and mixtures thereof.

The lotion composition of the present invention may further comprise a carrier. The carrier can help to deliver the omega-6 fatty acid of the present invention to the skin of the user of the wipe article. The carrier can be included in the compositions as an individual carrier or a combination of carrier ingredients. The carrier can be a liquid, solid, or semisolid carrier material, or a combination of these materials, and preferably forms a homogenous mixture or solution at selected processing temperatures for the resultant carrier system and at processing temperatures for combining the carrier with the cooling agents in formulating the lotion compositions herein. Processing temperatures for the carrier system typically do not exceed 90° C.

The lotion compositions of the present invention can comprise a carrier wherein the carrier may generally comprise any of the following ingredients: emollients, surfactants, preservatives. The composition may be an aqueous-based solution, a non aqueous-based solution, or an emulsion.

### Emollient

Emollients may (1) improve the glide of the substrate on the skin, by enhancing the lubrication and thus decreasing the abrasion of the skin, (2) hydrate the residues (for example, fecal residues or dried urine residues or menses), thus enhancing their removal from the skin, (3) hydrate the skin, thus reducing its dryness and irritation while improving its flexibility under the wiping movement, and (4) protect the skin from later irritation (for example, caused by the friction of an absorbent article) as the emollient is deposited onto the skin and remains at its surface as a thin protective layer.

Emollients may include silicone oils, functionalized silicone oils, hydrocarbon oils, fatty alcohols, fatty alcohol ethers, polysiloxanes, fatty acids, esters of monobasic and/or dibasic and/or tribasic and/or polybasic carboxylic acids with mono and polyhydric alcohols, polyoxyethylenes, polyoxypropylenes, mixtures of polyoxyethylene and polyoxypropylene ethers of fatty alcohols, and mixtures thereof. The emollients may be either saturated or unsaturated, have an aliphatic character and be straight or branched chained or contain alicyclic or aromatic rings.

A useful mixture of emollients is caprylic capric triglycerides in combination with Bis-PEG/PPG-16/16 PEG/PPG-16/16 dimethicone known as ABIL CARE™ 85 (available from Degussa Care Specialties of Hopewell, Va.).

Other suitable carrier compounds include petroleum-based hydrocarbons having from about 4 to about 32 carbon atoms, fatty alcohols having from about 12 to about 24 carbon atoms, polysiloxane compounds, fatty acid esters, alkyl ethoxylates, lower alcohols having from about 1 to about 6 carbon atoms, low molecular weight glycols and polyols, fatty alcohol ethers having from about 12 to about 28 carbon atoms in their fatty chain, lanolin and its derivatives, glyceride and its derivatives including acetoglycerides and ethoxylated glycerides of C12-C28 fatty acids, and mixtures thereof.

Other carriers suitable herein can include oils or fats such as natural oils or fats, or natural oil or fat derivatives, in particular of plant or animal origin. Non-limiting examples include apricot oil, babassu oil, castor oil, coconut oil, cod liver oil, hydrogenated corn oil, hydrogenated cottonseed oil, hazelnut oil, jojoba oil, macadamia oil, meadowfoam seed oil, mink oil, maring a oil, manila oil, mortierella oil, palm kernel oil, hydrogenated peanut oil, hydrogenated rapeseed oil, rose hip oil, hydrogenated safflower oil, hydrogenated soybean oil, hydrogenated sunflower oil, hydrogenated walnut oil, hydrogenated wheat germ oil, or the hardened derivatives thereof.

Suitable carriers further encompass waxes. As used herein, the term ‘wax’ refers to oil soluble materials that have a waxy constituency and have a melting point or range of above ambient temperature, in particular above 25° C. Waxes are materials that have a solid to semi-solid (creamy) consistency, crystalline or not, being of relative low viscosity a little above their liquefying point. Suitable waxes which can be incorporated into the lotion composition include animal, vegetable, mineral or silicone based waxes which may be natural or synthetic, and including mixtures thereof. Waxes can include but are not limited to: natural waxes from vegetal origin, such as bayberry wax, beeswax, candelilla wax, carnauba, ceresin, shea butter, cocoa butter, Japan wax, jojoba wax, lanolin wax, ouricury wax, mink wax, montan wax, rice bran wax, steryl dimethicone, fruit-derived waxes, such as orange wax, lemon wax, and the like; and waxes from animal origin such as beeswax, woolwax, bear fat, and the like. Natural waxes further comprise mineral waxes such as ceresin and ozokerite waxes. Synthetic waxes comprise petroleum-based waxes, such as certain carrier materials described hereinbefore, such as paraffin, vaseline, petrolatum, micro wax, and microcrystalline wax. Further suitable synthetic waxes are polyalkylene and polyethyleneglycol waxes, e.g. polyethylene wax; waxes based on chlorinated naphtalenes such as ‘Halowax’, synthetic hydrocarbon waxes, and the like, PEG-6 beeswax, PEG-8 beeswax, C30 alkyl dimethicone, synthetic beeswax, synthetic candelilla wax, synthetic carnuba wax, synthetic japan wax, synthetic jojoba wax, motan acid wax, motan wax, ouricury wax, rezowax, including mixtures thereof.

Other suitable carriers include materials that act as solidifying agents, including some of the materials described hereinbefore. Suitable solidifying agent(s) in the lotion compositions of the present invention can function to help solidify the composition so that the composition is a solid at room temperature and has a melting point of at least 32° C. The solidifying agent may also provide a tackiness to the composition that improves the transfer by adhesion to the skin of the wearer. Depending on the solidifying agent selected, the solidifying agent can also modify the mode of transfer so that the composition tends to fracture or flake off instead of actually rubbing off onto the skin of the wearer which can lead to improved transfer to the skin. The solidifying agent may further function as an emollient, occlusive agent, moisturizer, barrier enhancer, viscosity enhancer and combinations thereof. The solidifying agents can be selected from alkyl siloxanes, polymers, hydrogenated vegetable oils having a melting point of 35° C. or greater, fatty acid esters with a melting point of 35° C. or greater, alkyl hydroxystearates, branched esters, alkoxylated alcohols and alkoxylated carboxylic acid. Additionally, the solidifying agents can be selected from animal, vegetable and mineral waxes and alkyl silicones. Examples of suitable solidifying agents include, but are not limited to, the following: alkyl silicones, alkyl trimethylsilanes, beeswax, behenyl behenate, behenyl benzoate, C24-C28 alkyl dimethicone, C30 alkyl dimethicone, cetyl methicone, stearyl methicone, cetyl dimethicone, stearyl dimethicone, cerotyl dimethicone, candelilla wax, carnuba, synthetic carnuba, PEG-12 carnauba, cerasin, hydrogenated microcrystalline wax, jojoba wax, microcrystalline wax, lanolin wax, ozokerite, paraffin, synthetic paraffin, cetyl esters, behenyl behenate, C20-C40 alkyl behenate, C2-C5 lactate, cetyl palmitate, stearyl palmitate, isosteryl behenate, lauryl behenate, stearyl benzoate, behenyl isostearate, cetyl myristate, cetyl octanoate, cetyl oleate, cetyl ricinoleate, cetyl stearate, decyl oleate, di C2-C5 alkyl fumerate, dibehenyl fumerate, myristyl lactate, myristyl lignocerate, myristyl myristate, myristyl stearate, lauryl stearate, octyidodecyl stearate; octyidodecyl stearoyl stearate, oleyl arachidate, oleyl stearate, tridecyl behenate, tridecyl stearate, tridecyl stearoyl stearate, pentaerythrityl tetrabehenate, penteerythritylhydrogenated rosinate, pentaerythrityl distearate, pentaerythrityltetraabeite, penteerythrityl tetracocoate, penteerythrityl tetraperlargonate, pentserythrityl tetrastearate, ethylene vinyl acetate, polyethylene, hydrogenated cottonseed oil, hydrogenated vegetable oil, hydrogenated squalene, hydrogenated coconut oil, hydrogenated jojoba oil, hydrogenated palm oil, hydrogenated palm kernel oil, hydrogenated olive oil, polyamides, metal stearates and other metal soaps, C30-C60 fatty alcohols, C20+fatty amides, polypropylene, polystyrene, polybutane, polybutylene terephthalate, polydipentane, polypropylene, zinc stearate, dodecyl laurate, stearyl palmitate, octadecyl hexedecanoate, octadecyl palmitate, stearyl behenate, docosyl octanoate, tetradecyl-octadecanyl behenate, hexadecyl-cosanyl hexacosanate, shellac wax, glycol montanate, fluoranated waxes, C20-C40 alkyl hydroxystearyl stearate, and mixtures of such compounds.

The wipe article of the present invention can optionally further comprise essential oil materials that help to connote the benefits provided by the wipe article. Such essential oil materials can be applied to the wipe article separate from the lotion composition or can be made part of the lotion composition. Non-limiting examples of suitable essential oil materials include Acorns gramineus, Anthemis nobilis, Artemisia dracunculus, Basil, Bergamot, Calamintha sylvatica, Caraway, Cedarwood, Chamomile, Cineol, Cinnamon, Cinnamon bark, Citrus aurantium, Clove, Cypress, Dill, Eucalyptus, Eugenol, Frankincense, Galangol, Geranium, Ginger, Hibiscus, Hop, Jasmine, Juniper, Laurus nobilis, Lavender, Lemon balm, Lemongrass, Lemon, Limonene, Linalool, Linalyl acetate, Lippia alba, Marjoram, Melissa, Myrrh, Neroli, Nutmeg, Passiflora, Patchouli, Peppermint, Pinene, Rose, Rosewood, Rosemary, Sage, Sandalwood, Spearmint, Sweet Fennel, Sweet Orange, Tea Tree, Thyme, Valerian, Ylang ylang, Zadoary, Hibiscus, or mixtures thereof. Preferred essential oils associated with arousal include Cypress, Hibiscus, Juniper, Cineol, Citrus, Sweet Orange, and Rosemary. Preferred oils associated with a harmonizing effect include Lavender, Neroli, and Ylang ylang.

### Surfactant

The surfactant can be an individual surfactant or a mixture of surfactants. The surfactant may be a polymeric surfactant or a non-polymeric one. The surfactant may be employed as an emulsifier. The surfactant, when present, may be employed in an amount effective to emulsify the emollient and any other non-water-soluble oils that may be present in the composition.

The composition may include one or more surfactants. The surfactant or combinations of surfactants may be mild, which means that the surfactants provide sufficient cleansing or detersive benefits but do not overly dry or otherwise harm or damage the skin.

A wide variety of surfactants are useful herein and include those selected from the group consisting of anionic surfactants, nonionic surfactants, cationic surfactants, amphoteric surfactants, zwitterionic surfactants, and mixtures thereof.

A wide variety of anionic surfactants are useful herein. Non-limiting examples of anionic surfactants include those selected from the group consisting of sarcosinates, sulfates, sulfonates, isethionates, taurates, phosphates, lactylates, glutamates, and mixtures thereof. Amongst the isethionates, the alkoyl isethionates are useful, and amongst the sulfates, the alkyl and alkyl ether sulfates are useful. Other anionic materials useful herein are soaps (i.e., alkali metal or amine salts, e.g., sodium, potassium or triethanol amine salts) of fatty acids, typically having from about 8 to about 24 carbon atoms.

Nonionic surfactants useful herein include, but are not limited to, those selected from the group consisting of alkyl glucosides, alkyl polyglucosides, polyhydroxy fatty acid amides, alkoxylated fatty acid esters, alkoxylated fatty alcohol ethers, sucrose esters, amine oxides, and mixtures thereof.

Suitable amphoteric or zwitterionic surfactants for use in the compositions herein include those which are known for use in hair care or other personal care cleansing. Amphoteric surfactants suitable for use in the present compositions are well known in the art and include those surfactants broadly described as derivatives of aliphatic secondary and tertiary amines in which the aliphatic radical can be straight or branched chain and wherein one of the aliphatic substituents contains from about 8 to about 18 carbon atoms and one contains an anionic water solubilizing group such as carboxy, sulfonate, sulfate, phosphate, or phosphonate. Useful amphoteric surfactants include, but are not limited to, the group consisting of cocoamphoacetate, cocoamphodiacetate, lauroamphoacetate, lauroamphodiacetate, and mixtures thereof.

Zwitterionic surfactants suitable for use herein include those surfactants broadly described as derivatives of aliphatic quaternary ammonium, phosphonium, and sulfonium compounds, in which the aliphatic radicals can be straight or branched chain, and wherein one of the aliphatic substituents contains from about 8 to about 18 carbon atoms and one contains an anionic group such as carboxy, sulfonate, sulfate, phosphate or phosphonate. Useful zwitterionic detersive surfactants are the betaines, amphoacetates and sulfobetaines, e.g., cocoamidopropylbetaine, sodiumlaurylamphoacetate and cocoamidopropylhydroxysultaine.

The wipe article or lotion composition of the present invention can further comprise a cooling agent. Suitable cooling agents are described in US 2004/0081680 A1 and US 2009/0240223 A1.

The wipe of the present invention can contain an effective amount of the lotion composition. As used herein, the term “effective amount of a lotion composition” refers to an amount of a particular lotion composition which, when applied to a wipe, will be effective in transferring omega-6 fatty acid to the skin of the wearer. The effective amount of a lotion composition will depend, to a large extent, on the particular lotion composition used.

In preparing lotioned wipe articles according to the present invention, the lotion composition can be applied to the surface of the wipe article. Any of a variety of application methods that evenly distribute the lotion composition can be used. Suitable methods include spraying, printing (e.g., flexographic printing), coating (e.g., gravure coating), extrusion, or combinations of these application techniques, e.g. spraying the lotion composition on a rotating surface, such as a calender roll, that then transfers the composition to the outer surface of the topsheet. Lotion compositions of the present invention can be applied by printing methods, or continuous spray or extrusion as is known in the art, or as is described in U.S. Pat. No. 5,968,025.

The manner of applying the lotion composition to the surface of the wipe article can be such that the wipe article does not become saturated with the lotion composition. In another embodiment, the wipe article may be saturated with the lotion composition. Saturation of the wipe article is not required to obtain the therapeutic and/or protective lotion benefits. Particularly suitable application methods will apply the lotion composition primarily to the outer surface of the wipe article.

The amount of lotion composition within each wet wipe may vary depending upon the type of material being used to provide the wet wipe or wipe-type product, the type of container being used to store the wet wipes, and the desired end use of the wet wipe. Generally, each wet wipe or wipe-type product can contain from about 100 to about 600 weight percent and desirably from about 250 to about 450 weight percent liquid based on the dry weight of the wipe for improved wiping.

The amount of lotion composition within each dry wipe may vary depending upon the type of material being used to provide the dry wipe or wipe-type product, the type of container being used to store the dry wipes, and the desired end use of the dry wipe. Generally, each dry wipe or wipe-type product can contain from about 0.5% to about 250% by weight of the substrate, preferably, from about 0.5% to about 100% by weight of the substrate, and most preferably from about 0.5% to about 25% by weight of the substrate.

The lotion composition may be applied to the entire surface of the wipe article or portions thereof. The lotion composition can be applied in a stripe aligned with and centered on the longitudinal centerline of the wipe article. The lotion composition can be applied in a plurality of stripes having uniform or non-uniform widths. Alternatively the lotion can be aligned with and centered in apposition to the longitudinal centerline.

In certain embodiments, the lotion be applied in a plurality of stripes parallel to the longitudinal axis of the wipe article. This allows for transfer of the lotion to a broader area of the vulva and improved fluid handling of the wipe article.

The lotion composition can also be applied nonuniformly to the outer surface of the wipe article. By “nonuniform” is meant that the amount, pattern of distribution, etc. of the lotion composition can vary over the wipe article surface. For example, some portions of the treated surface of the wipe article can have greater or lesser amounts of lotion composition, including portions of the surface that do not have any lotion composition on it. For example, the lotion composition can be applied on one region of the wipe article in the shape of a rectangle and/or a circle, and/or as mutliplicity of dots.

The lotion composition can be applied to the surface at any point during assembly. For example, the lotion composition can be applied to the wipe article before it has been packaged.

The lotion composition may be applied from a melt thereof to the wipe article. Since the lotion composition will typically melt at significantly above ambient temperatures, it is usually applied as a heated coating. Typically, the lotion composition is heated to a temperature in the range from about 35° C. to about 100° C., preferably from 40° C. to about 90° C., prior to being applied. Once the melted lotion composition has been applied, it is allowed to cool and solidify to form solidified coating or film on the surface of the topsheet or other component. Preferably, the application process is designed to aid in the cooling/set up of the lotion.

In certain embodiments, the wipe article can be joined to a sanitary napkin, pantiliner, tampon, or other suitable feminine hygiene article, (for example, as described in detail in U.S. Pat. No. 5,569,230, U.S. Pat. No. 6,911,022 or WO 03/057122 A1). In another embodiment, the lotion composition can be provided as a stand-alone product in the form of a cream product that can be applied to the wipe article or to the skin by hand (for example, as described in detail in U.S. Pat. No. 5,948,416). In another embodiment, the lotion composition can be provided as a stand-alone product in the form of a spray or mousse product that can be sprayed onto the wipe article or the skin by the wearer of an absorbent article (for example, as described in detail in U.S. Pat. No. 4,708,813).

### Method of Improving Skin Barrier Function of Vulvar Skin

The present invention further encompasses a method of improving skin barrier function of vulvar skin, said method comprising the step of contacting said vulvar skin with a wipe article comprising a body facing surface and a garment facing surface, wherein omega-6 fatty acid is disposed on said body facing surface of said wipe article. Improvement in skin barrier function can be exhibited by improved skin lipid composition, improved skin moisturization, or the like.

The amount of lotion composition within each wet wipe may vary depending upon the type of material being used to provide the wet wipe or wipe-type product, the type of container being used to store the wet wipes, and the desired end use of the wet wipe. Generally, each wet wipe or wipe-type product can contain from about 100 to about 600 weight percent and desirably from about 250 to about 450 weight percent liquid based on the dry weight of the wipe for improved wiping.

The amount of lotion composition within each dry wipe may vary depending upon the type of material being used to provide the dry wipe or wipe-type product, the type of container being used to store the dry wipes, and the desired end use of the dry wipe. Generally, each dry wipe or wipe-type product can contain from about 0.5% to about 250% by weight of the substrate, preferably, from about 0.5% to about 100% by weight of the substrate, and most preferably from about 0.5% to about 25% by weight of the substrate.

Any suitable method can be used in determining the amount of a lotion composition described herein that is transferred to the skin. Examples of specific methods for the calculation of transfer amounts of lotion compositions include gas chromatography and other quantitative analytical procedures that involve the analysis of in vivo skin analog materials. A suitable gas chromatographic procedure is more fully described in WO 99/45973, Donald C. Roe et al, published Sep. 16, 1999.

The present invention further encompasses the use of a wipe article comprising omega-6 fatty acid, such as those described herein, for improving the skin barrier function of vulvar skin.

The following are non-limiting examples of the present invention. In the Examples, “QS” refers herein to “quantum sufficit” and is a sufficient percentage of water added to the composition to bring the overall composition to 100%.

Dry Wipes

The compositions exemplified hereinbelow in Table 1 are representative of the lotion compositions of the present invention for a dry wipe. The lotion systems are generally prepared by combining, by weight, the components in the first table below under heat until molten. Numbers below represent weight percents.

| Component | Ex 1 | Ex 2 | Ex 3 | Ex 4 | Ex. 5 |
|---|---|---|---|---|---|
| SEFA* Cottonate | 38 | — | — | 9 | — |
| SEFA* Behenate | 12 | — | — | — | — |
| Petrolatum | 10 | 20 | 20 | 10 | 33 |
| Tribehenin | 5 | — | — | — | — |
| Lipex ® Omega Passiflora | 10 | 60 | 6 | 30 | — |
| Lipex ® Omega 3/6 | — | — | 50 | | 40 |
| High Oleic Canola Oil | — | — | — | 30 | 13 |
| C₁₀-C₃₀ | 25 | — | — | — | 15 |
| Cholesterol/Lanosterol Esters | | | | | |
| Synthetic Beeswax | — | 3 | 3 | 3 | 3 |
| Polyethylene Wax | — | 9 | 9 | 9 | 9 |
| Paraffin | — | 8 | 12 | 8 | — |
| Amount added to substrate (g) | 0.25 | 0.25 | 0.25 | 0.35 | 0.1 |

Optionally, dry wipes may also be prepared according to the following method. First, one prepares a surfactant according to the following composition at room temperature.

| | Weight Percent | | | | |
|---|---|---|---|---|---|
| Ingredients | Ex. 6 | Ex. 7 | Ex. 8 | Ex. 9 | Ex. 10 |
| Water | QS 100 | QS 100 | QS 100 | QS 100 | QS 100 |
| Polyquaternium-10 | — | 0.25 | — | — | — |
| PEG 14M | — | 0.5 | 0.5 | — | — |
| Hydroxypropyltrimonium | — | — | — | — | 0.25 |
| Chloride | | | | | |
| Hydroxyelthylcellulose | 0.25 | — | — | — | 0.5 |
| Guar Gum | 0.25 | — | — | — | — |

The following components are added to the surfactant mixture:

| | | | | | |
|---|---|---|---|---|---|
| Disodium EDTA | 0.1 | 0.1 | 0.1 | 0.1 | 0.1 |
| Sodium Lauroyl Sarcosinate | 3.33 | 3.33 | 3.33 | 3.33 | 3.33 |
| Sodium Lauroamphoacetate | — | — | — | — | 3.33 |
| Cocamidopropyl Betaine | 3.33 | 3.33 | 3.33 | 3.33 | — |
| Decyl Polyglucoside | 3.33 | 3.33 | 3.33 | 3.33 | 3.33 |
| Methyl Paraben | 0.25 | 0.25 | 0.25 | 0.25 | 0.25 |
| Phenoxyethanol | 0.3 | 0.3 | 0.3 | 0.3 | 0.3 |
| Benzyl Alcohol | 0.3 | 0.3 | 0.3 | 0.3 | 0.3 |
| Glycerin | — | — | — | — | 3 |
| Urea | | | | — | 1 |

In a separate mixing vessel, the following components are added. The combination is mixed (with heat to 40° C. as necessary) until propyl paraben is dissolved.

| | | | | | |
|---|---|---|---|---|---|
| Water | 2 | 2 | 2 | 2 | 2 |
| Butylene Glycol | 2 | 2 | 2 | 2 | 2 |
| Propyl Paraben | 0.15 | 0.15 | 0.15 | 0.15 | 0.15 |

This mixture is added to the first mixing vessel. About 1.5-2.5 g of the resultant mixture are added to a non-woven substrate and then dried.

Wet Wipe

The compositions exemplified hereinbelow in Table 1 are representative of the lotion compositions of the present invention for a wet wipe. The lotion compositions of Examples 1 through 7 are contacted as described earlier with a substrate such as Fibrella 3160, a 58 grams/m² nonwoven comprising a blend of 40% viscose fibers and 60% polypropylene fibers as is available from Suominen of Tampere, Finland or any other substrate deemed suitable for use. Numbers represent weight percents.

| Component | Ex. 1 | Ex. 2 | Ex. 3 | Ex. 4 | Ex. 5 | Ex. 6 | Ex. 7 |
|---|---|---|---|---|---|---|---|
| Water | Q.S | Q.S | Q.S | Q.S | Q.S | Q.S | Q.S |
| Disodium EDTA¹¹ | 0.100 | 0.100 | 0.100 | 0.100 | 0.100 | 0.100 | 0.100 |
| Iodopropynyl | 0.009 | 0.009 | 0.009 | 0.009 | 0.009 | 0.009 | 0.009 |
| Butylcarbamate | | | | | | | |
| (IPBC)¹ | | | | | | | |
| Benzyl alcohol² | 0.500 | 0.500 | 0.500 | 0.500 | 0.500 | 0.500 | 0.500 |
| Suttocide ® A | 0.150 | 0.150 | 0.150 | 0.150 | 0.150 | 0.150 | 0.150 |
| (50% soln)³ | | | | | | | |
| Phenoxyethanol¹¹ | 0.000 | 0.000 | 0.000 | 0.000 | 0.000 | 0.000 | 0.000 |
| Methyl Paraben¹¹ | 0.000 | 0.000 | 0.000 | 0.000 | 0.000 | 0.000 | 0.000 |
| Ethyl Paraben¹¹ | 0.000 | 0.000 | 0.000 | 0.000 | 0.000 | 0.000 | 0.000 |
| Propyl Paraben¹¹ | 0.000 | 0.000 | 0.000 | 0.000 | 0.000 | 0.000 | 0.000 |
| Xanthan Gum¹² | 0.180 | 0.180 | 0.180 | 0.180 | 0.180 | 0.180 | 0.180 |
| Poloxamer 124¹⁰ | 0.050 | 0.050 | 0.050 | 0.050 | 0.050 | 0.050 | 0.050 |
| Polysorbate 20¹⁰ | 0.030 | 0.030 | 0.030 | 0.030 | 0.030 | 0.030 | 0.030 |
| Abil Care 85⁴ | 0.100 | 0.100 | 0.100 | 0.100 | 0.100 | 0.100 | 0.100 |
| Monobasic Sodium | 0.000 | 0.000 | 0.000 | 0.000 | 0.000 | 0.000 | 0.000 |
| Phosphate¹² | | | | | | | |
| Citric Acid⁵ | 0.055 | 0.055 | 0.055 | 0.055 | 0.055 | 0.055 | 0.055 |
| Perfume | 0.070 | 0.070 | 0.070 | 0.070 | 0.070 | 0.070 | 0.070 |
| Dimethicone⁹ | 0.000 | 0.000 | 0.000 | 0.000 | 0.000 | 0.000 | 0.000 |
| Cyclomethicone⁹ | 0.000 | 0.000 | 0.000 | 0.000 | 0.000 | 0.000 | 0.000 |
| Lipex ® Omega | 0.500 | 2.000 | 5.000 | 0.500 | 0.000 | 2.000 | 0.000 |
| Passiflora⁷ | | | | | | | |
| Lipex ® Omega 3/6⁷ | 0.000 | 0.000 | 0.000 | 0.500 | 0.500 | 2.000 | 0.500 |
| High Oleic Canola | 0.000 | 0.000 | 0.000 | 0.000 | 0.000 | 2.000 | 0.500 |
| Oil⁸ | | | | | | | |
| Niacinamide⁵ | 2.000 | 2.000 | 0.000 | 0.000 | 2.000 | 0.000 | 2.000 |
| Glycerin⁶ | 1.000 | 0.000 | 1.000 | 0.000 | 0.500 | 0.000 | 1.000 |
| Panthenol⁵ | 0.500 | 0.500 | 0.500 | 0.000 | 0.500 | 0.000 | 0.500 |

| Component | Ex. 8 | Ex. 9 | Ex. 10 | Ex. 11 | Ex. 12 |
|---|---|---|---|---|---|
| Water | Q.S | Q.S | Q.S | Q.S | Q.S |
| Disodium EDTA¹¹ | 0.000 | 0.000 | 0.100 | 0.100 | 0.000 |
| Iodopropynyl Butylcarbamate | 0.000 | 0.000 | 0.009 | 0.009 | 0.000 |
| (IPBC)¹ | | | | | |
| Benzyl alcohol² | 0.000 | 0.000 | 0.500 | 0.500 | 0.000 |
| Suttocide ® A (50% soln)³ | 0.000 | 0.000 | 0.150 | 0.150 | 0.000 |
| Phenoxyethanol¹¹ | 0.800 | 0.800 | 0.000 | 0.000 | 0.800 |
| Methyl Paraben¹¹ | 0.150 | 0.150 | 0.000 | 0.000 | 0.150 |
| Ethyl Paraben¹¹ | 0.050 | 0.050 | 0.000 | 0.000 | 0.050 |
| Propyl Paraben¹¹ | 0.050 | 0.050 | 0.000 | 0.000 | 0.050 |
| Xanthan Gum¹² | 0.180 | 0.180 | 0.000 | 0.000 | 0.000 |
| Poloxamer 124¹⁰ | 1.000 | 1.000 | 0.050 | 0.050 | 1.000 |
| Polysorbate 20¹⁰ | 0.500 | 0.500 | 0.030 | 0.030 | 0.500 |
| Abil Care 85⁴ | 0.100 | 0.100 | 0.000 | 0.000 | 0.000 |
| Monobasic Sodium | 0.180 | 0.180 | 0.000 | 0.000 | 0.180 |
| Phosphate¹² | | | | | |
| Citric Acid⁵ | 0.000 | 0.000 | 0.055 | 0.055 | 0.000 |
| Perfume | 0.070 | 0.070 | 0.070 | 0.070 | 0.070 |
| Dimethicone⁹ | 0.000 | 0.100 | 0.200 | 0.200 | 0.100 |
| Cyclomethicone⁹ | 0.000 | 0.050 | 0.100 | 0.100 | 0.050 |
| Lipex ® Omega Passiflora⁷ | 0.200 | 0.000 | 0.000 | 0.000 | 3.000 |
| Lipex ® Omega 3/6⁷ | 0.200 | 4.000 | 5.000 | 0.000 | 0.000 |
| High Oleic Canola Oil⁸ | 0.000 | 0.000 | 0.000 | 0.500 | 0.000 |
| Niacinamide⁵ | 0.000 | 0.000 | 0.000 | 2.000 | 0.000 |
| Glycerin⁶ | 0.000 | 0.000 | 0.500 | 0.500 | 0.000 |
| Panthenol⁵ | 0.000 | 0.000 | 0.500 | 0.000 | 0.000 |

¹IPBC available from Clariant UK, Ltd.; Leeks, United Kingdom
²Benzyl Alcohol available from Symrise, Inc.; Teterboro, NJ
³Suttocide ® A (50% soln) available from International Specialty Products; Wayne, NJ
⁴Abil Care 85 available from Evonik Goldschmidt; Hopewell, VA
⁵Niacinamide, Panthenol, and Citiric acid available from DSM Nutritional; Parsippany, NJ
⁶Glycerin available from Procter and Gamble; Cincinnati, OH
⁷Omega Oils available from Aarhus Karlshamn; Karlshamn, Sweden
⁸Oleic Canola Oil available from Clear Valley ® 75 High Oleic Canola Oil from Cargill
⁹Silicones Available from Dow Corning; Midland, MI
¹⁰Poloxamer and Polysorbate available from BASF Inc.; Florham Park, NJ
¹¹Parabens and Phenoxyethanol available from DeWolf Chemical; East Providence, RI
¹²Monobasic Sodium Phosphate available from Charkit Chemical; Norwalk, CT

In preparing the solutions representative of examples 1-9 all materials are blended in suitable mixing vessels at room temperature using a propeller type mixer. Final blends are mixed using a homogenizer. Into one vessel (oil phase) is added Abil Care, 50% of the Lipex Omega Passiflora, Lipex Omega 3/6, and/or High Oleic Canola Oil, and preservative system. Into another vessel (water phase) are added the water phase ingredients of 90% water, non-ionic surfactants, glycerin, niaciamide, and panthenol. The water phase is then added to oil phase and the combined mixtures are homogenized. The xanthan gum predispered in the remaining 50% oil is added and the emulsion homogenized again. The mixture is neutralized to pH 5-5.5 and qs with water and homogenized. If an oil based fragrance is used, it is added to the oil phase step. Otherwise, it is added to the water phase step.

In preparing solutions representative of examples examples 10-12, all materials are blended in suitable mixing vessels at room temperature using a propeller type mixer. Final blends are mixed using a homogenizer. Into one vessel (water phase) are added 90% of the water, the emulsifiers, glycerin, niaciamide, and panthenol. Into another vessel (oil phase) are added dimethicone, cyclomethicone, Lipex Omega Passiflora, Lipex Omega 3/6, and/or High Oleic Canola Oil, and the preservative system. The water phase is then added to the oil phase and the combined mixtures are homogenized. The mixture is neutralized to pH 5-5.5 and qs with water and homogenized. If an oil based fragrance is used, it is added to the oil phase step. Otherwise, it is added to the water phase step.

The dimensions and values disclosed herein are not to be understood as being strictly limited to the exact numerical values recited. Instead, unless otherwise specified, each such dimension is intended to mean both the recited value and a functionally equivalent range surrounding that value. For example, a dimension disclosed as “40 mm” is intended to mean “about 40 mm.”

Every document cited herein, including any cross referenced or related patent or application, is hereby incorporated herein by reference in its entirety unless expressly excluded or otherwise limited. The citation of any document is not an admission that it is prior art with respect to any invention disclosed or claimed herein or that it alone, or in any combination with any other reference or references, teaches, suggests or discloses any such invention. Further, to the extent that any meaning or definition of a term in this document conflicts with any meaning or definition of the same term in a document incorporated by reference, the meaning or definition assigned to that term in this document shall govern.

While particular embodiments of the present invention have been illustrated and described, it would be obvious to those skilled in the art that various other changes and modifications can be made without departing from the spirit and scope of the invention. It is therefore intended to cover in the appended claims all such changes and modifications that are within the scope of this invention.

1. A method of improving skin barrier function of vulvar skin, said method comprising the step of contacting said vulvar skin with a wipe wherein omega-6 fatty acid is disposed on a surface of said wipe article.

2. The method of claim 1, wherein a lotion composition comprises (a) an oil material comprising said omega-6 fatty acid and (b) a carrier, wherein said lotion composition is disposed on said wipe article.

3. The method of claim 1, wherein a lotion composition comprises from about 0.003% to about 35%, by weight of said lotion composition, of said omega-6 fatty acid, and from about 0.01% to about 56%, by weight of said lotion composition, of oleic acid.

4. The method of claim 2, wherein said oil material is selected from the group consisting of a mixture of passiflora incarnata seed oil and vegetable oil, a mixture of camelina sativa seed oil and vegetable oil, a mixture of evening primrose oil and vegetable oil, high oleic canola oil, and mixtures thereof.

5. The method of claim 2, wherein said oil material comprises at least about 3%, by weight of the oil material, of omega-6 fatty acid.

6. The method of claim 2, wherein said lotion composition further comprises an anti-oxidant material selected from the group consisting of α-tocopherol, β-tocopherol, γ-tocopherol, δ-tocopherol, α-tocotrienol, γ-tocotrienol, δ-tocotrienol, sesamolin, sesamin, catechin, and mixtures thereof.

7. The method of claim 3, wherein said lotion composition comprises from about 0.003% to about 35%, by weight of said lotion composition, of said omega-6 fatty acid.

8. The method of claim 2, wherein said lotion composition further comprises an essential oil selected from the group consisting of Acorns gramineus, Anthemis nobilis, Artemisia dracunculus, Basil, Bergamot, Calamintha sylvatica, Caraway, Cedarwood, Chamomile, Cinnamon, Cinnamon bark, Citrus aurantium, Clove, Cypress, Dill, Eucalyptus, Eugenol, Frankincense, Galangol, Geranium, Ginger, Hop, Jasmine, Laurus nobilis, Lavender, Lemon balm, Lemongrass, Lemon, Limonene, Linalool, Linalyl acetate, Lippia alba, Marjoram, Melissa, Myrrh, Neroli, Nutmeg, Passiflora, Patchouli, Peppermint, Pinene, Rose, Rosewood, Rosemary, Sage, Sandalwood, Spearmint, Sweet Fennel, Sweet Orange, Thyme, Valerian, Ylang ylang, and mixtures thereof.

9. The method of claim 2, wherein said oil material has an oil stability index of at least about 10 hours.

10. A wipe comprising:
a non-woven material, said non-woven material releasably carrying a lotion composition disposed on one or more layers of said wipe article, said lotion composition comprising (a) an oil material comprising at least about 3%, by weight of said oil material, of omega-6 fatty acid, wherein said oil material has an oil stability index of at least about 10 hours and is selected from the group consisting of a mixture of passiflora incarnata seed oil and vegetable oil, a mixture of camelina sativa seed oil and vegetable oil, a mixture of evening primrose oil and vegetable oil, high oleic canola oil, and mixtures thereof, and (b) a carrier.

11. The wipe article of claim 10, wherein said oil material further comprises at least about 10%, by weight of said oil material, of oleic acid.

12. The wipe article of claim 10, wherein said lotion composition further comprises an anti-oxidant material selected from the group consisting of α-tocopherol, β-tocopherol, γ-tocopherol, δ-tocopherol, α-tocotrienol, γ-tocotrienol, δ-tocotrienol, sesamolin, sesamin, catechin, and mixtures thereof.

13. The wipe article of claim 10, wherein said lotion composition further comprises an essential oil selected from the group consisting of Acorns gramineus, Anthemis nobilis, Artemisia dracunculus, Basil, Bergamot, Calamintha sylvatica, Caraway, Cedarwood, Chamomile, Cinnamon, Cinnamon bark, Citrus aurantium, Clove, Cypress, Dill, Eucalyptus, Eugenol, Frankincense, Galangol, Geranium, Ginger, Hop, Jasmine, Laurus nobilis, Lavender, Lemon balm, Lemongrass, Lemon, Limonene, Linalool, Linalyl acetate, Lippia alba, Marjoram, Melissa, Myrrh, Neroli, Nutmeg, Passiflora, Patchouli, Peppermint, Pinene, Rose, Rosewood, Rosemary, Sage, Sandalwood, Spearmint, Sweet Fennel, Sweet Orange, Thyme, Valerian, Ylang ylang, and mixtures thereof.

14. The wipe article of claim 10, wherein said oil material has an oil stability index value of at least about 14 hours.

15. A wipe article comprising:
a non-woven material, said non-woven material releasably carrying a lotion composition disposed on one or more layers of said wipe article, said lotion composition comprising: (a) at least about 0.003%, by weight of said lotion composition, of omega-6 fatty acid, (b) at least about 0.01%, by weight of said lotion composition, of oleic acid, and (c) a carrier.

16. The wipe article of claim 15, wherein said oil material is selected from the group consisting of a mixture of passiflora incarnata seed oil and vegetable oil, a mixture of camelina sativa seed oil and vegetable oil, a mixture of evening primrose oil and vegetable oil, high oleic canola oil, and mixtures thereof.

17. The wipe article of claim 15, wherein said lotion composition further comprises an anti-oxidant material selected from the group consisting of α-tocopherol, β-tocopherol, γ-tocopherol, δ-tocopherol, α-tocotrienol, γ-tocotrienol, δ-tocotrienol, sesamolin, sesamin, catechin and mixtures thereof.

18. The wipe article of claim 15, wherein said lotion composition further comprises an essential oil selected from the group consisting of Acorns gramineus, Anthemis nobilis, Artemisia dracunculus, Basil, Bergamot, Calamintha sylvatica, Caraway, Cedarwood, Chamomile, Cinnamon, Cinnamon bark, Citrus aurantium, Clove, Cypress, Dill, Eucalyptus, Eugenol, Frankincense, Galangol, Geranium, Ginger, Hop, Jasmine, Laurus nobilis, Lavender, Lemon balm, Lemongrass, Lemon, Limonene, Linalool, Linalyl acetate, Lippia alba, Marjoram, Melissa, Myrrh, Neroli, Nutmeg, Passiflora, Patchouli, Peppermint, Pinene, Rose, Rosewood, Rosemary, Sage, Sandalwood, Spearmint, Sweet Fennel, Sweet Orange, Thyme, Valerian, Ylang ylang, and mixtures thereof.

19. The wipe article of claim 15, wherein said carrier is selected from the group consisting of silicone oils, functionalized silicone oils, polysiloxanes, hydrocarbon oils, naturals fats and oils, petroleum-based hydrocarbons, fatty alcohols, fatty alcohol ethers, fatty acids, esters of monobasic and/or dibasic and/or tribasic and/or polybasic carboxylic acids with mono and polyhydric alcohols, polyoxyethylenes, polyoxypropylenes, mixtures of polyoxyethylene and polyoxypropylene ethers of fatty alcohols, surfactants, and mixtures thereof.

20. A method of improving skin barrier function, lipid composition, or moisturization of vulvar skin, said method comprising the step of contacting said vulvar skin with a wipe article, wherein the lotion of claim 3 is disposed on a surface of said wipe article.
